(19) United States
(12) Patent Application Publication
(10) Pub. No.: US 2019/0125509 A1
(43) Pub. Date: May 2, 2019

(54) METHOD AND APPARATUS FOR MONITORING ANIMALS

(71) Applicant: ANIMAL APPS PTY LTD, Warragul, Victoria (AU)

(72) Inventor: Thomas Luke Hotchkin, Warragul, Victoria (AU)

(21) Appl. No.: 16/095,660

(22) PCT Filed: May 3, 2017
(86) PCT No.: PCT/AU2017/050405

§ 371 Date: Oct. 22, 2018

(30) Foreign Application Priority Data
May 5, 2016 (AU) 2016901656
Nov. 23, 2016 (AU) 2016904813

(51) Int. Cl. A61D 17/00; A61B 5/11; A61B 5/00; H04W 4/029; A01K 29/00

(52) U.S. Cl. CPC A61D 17/008; A61B 5/1116; A61B 5/0022; A61B 5/1128; H04W 4/12; H04W 4/029; A01K 29/005; A61B 2562/0204; A61B 5/6898

(57) ABSTRACT

A method of operating a smartphone for monitoring an animal, the method comprising the steps of: monitoring a position and orientation of the smartphone; determining that a detection event indicative of the animal lying down from a standing position has occurred corresponding to a change in position and/or a change in orientation satisfying a predetermined criteria; and in response, communicating, at least in part via a public mobile telecommunications communication network, to one or more receiver devices a message indicative of identification of a detection event, and corresponding device and system.

### FIELD OF THE INVENTION

The invention generally relates to monitoring animals, for example, mares.

### BACKGROUND TO THE INVENTION

Owners of animals often have an interest the health of the animals. For example, horse breeders will typically be present when a mare is giving birth to a foal (“foaling”), in order to provide any assistance that should be required to minimise the chances of a problems occurring during birth. However, the exact time that a mare will give birth is almost impossible to predict.

There exist products designed to alert a breeder when a mare is about to give birth. These existing products, however, utilise specially designed equipment for the task, which can add unnecessary expense and complexity. It would therefore be desirable to provide a system for monitoring mares in order to provide an alert when foaling is about to occur that does not require specially designed equipment.

As another example, a concerned owner may wish to be able to monitor an unwell animal.

### SUMMARY OF THE INVENTION

According to an aspect of the invention, there is provided a method of operating a smartphone for monitoring an animal, the method comprising the steps of: monitoring a position and orientation of the smartphone; determining that a detection event indicative of the animal lying down from a standing position has occurred corresponding to a change in position and/or a change in orientation satisfying a predetermined criteria; and in response, communicating, at least in part via a public mobile telecommunications communication network, to one or more receiver devices a message indicative of identification of a detection event. The animal may be a mare.

In an embodiment, the method in response to determining that the detection event has occurred further comprising the steps of the smartphone: obtaining image data; and communicating said image data to the receiver device and/or one or more other devices. The image data may be video data. The image data may be a single photograph image. The image data may be obtained as a sequence of photograph images, wherein each subsequent photograph image within the sequence is obtained and communicated after at least a predetermined time has elapsed since the preceding image was obtained and communicated. Optionally, the method further comprises the step of activating a light of the smartphone. The light may be activated such that the image data is captured when the light is activated. The light may be activated in response to reception of a light instruction message comprising an instruction to activate the light from a receiver device and/or another device in communication with the smartphone. In this case, the method may further comprise the step of deactivating the light in response to reception of a light instruction message comprising an instruction to deactivate the light from a receiver device and/or another device in communication with the smartphone.

Optionally, the method in response to determining that the detection event has occurred further comprises the steps of the smartphone: obtaining audio data; and communicating said audio data to the receiver device and/or one or more other devices.

Preferably, the method further comprises the steps of: configuring the smartphone with at least the predetermined criteria; and initiating the smartphone in response to a user input, and then subsequently monitoring the position and orientation of the smartphone.

The smartphone may be configured to communicate with the receiver device in response to reception of a user input providing an identification of the receiver device.

Optionally, the predetermined criteria include: a minimum change in height of the smartphone; and/or a minimum change in orientation of the smartphone.

Optionally, the message is only communicated if the smartphone determines that the detection event has occurred for a predetermined minimum timespan.

Typically, the message is communicated from the smartphone using, at least in part, a mobile broadband protocol, for example selected from one or more of: W-CDMA, HSPA, CDMA2000, GSM EDGE-Evolution, HSPA+, LTE, and LTE-Advanced.

Preferably, the method further comprises the step of generating additional information in response to determining the detection event, and including the additional information into the message. The additional information may comprise GPS location information.

The method may further comprise the steps of: receiving auxiliary information, at the smartphone, from at least one local auxiliary device; and communicating the auxiliary information to at least one receiver device and/or at least one other device. Optionally, information related to the animal is received from at least one auxiliary device. Also optionally, information related to the environment of the animal is received from at least one auxiliary device. The method may further comprise the step of the smartphone processing the auxiliary information. At least one auxiliary device may be selected from: heart rate monitor; ambient temperature sensor; animal temperature sensor; and auxiliary camera.

According to another aspect of the present invention, there is provided a method of operating a smartphone for monitoring an animal, the method comprising the steps of: receiving auxiliary information from one or more local auxiliary devices; determining that the received auxiliary information meets predetermined auxiliary criteria associated with the auxiliary information; and in response, communicating, at least in part via a public mobile telecommunications communication network, to one or more receiver devices a message indicative of identification of the predetermined auxiliary criteria being met.

According to another aspect of the present invention, there is provided a smartphone, comprising a processor interfaced with a wireless communication module, a position sensor and/or an orientation sensor, the smartphone configured to execute a program configured to cause the smartphone to: monitor a position and orientation of the smartphone when attached to an animal; determine that a detection event indicative of the animal lying down from a standing position has occurred corresponding to a change in position and/or a change in orientation satisfying a predetermined criteria; and in response, communicate, at least in part via a public mobile telecommunications communication network, to one or more receiver devices a message indicative of identification of a detection event. The animal may be a mare.

In an embodiment, the program is further configured to cause the smartphone to: obtain image data from a camera of the smartphone; and communicate said image data to the receiver device and/or one or more other devices.

The image data may be video data. The image data may be a single photograph image. The image data may be obtained as a sequence of photograph images, wherein each subsequent photograph image within the sequence is obtained and communicated after at least a predetermined time has elapsed since the preceding image was obtained and communicated.

Optionally, the smartphone further comprises a light, and the program is configured to activate and deactivate the light. The light may be activated such that the image data is captured when the light is activated. The program may be configured to activate the light in response to reception of a light instruction message comprising an instruction to activate the light from a receiver device and/or another device in communication with the smartphone. In this case, the program may be configured to deactivate the light in response to reception of a light instruction message comprising an instruction to deactivate the light from a receiver device and/or another device in communication with the smartphone.

Optionally, the program is further configured to cause the smartphone to: obtain audio data; and communicate said audio data to the receiver device and/or one or more other devices.

Preferably, the program is further configured to cause the smartphone to: configure the smartphone with at least the predetermined criteria; and initiate the smartphone in response to a user input, and then subsequently monitor the position and orientation of the smartphone.

The smartphone may be configured to communicate with the receiver device in response to reception of a user input providing an identification of the receiver device.

Optionally, the predetermined criteria include: a minimum change in height of the smartphone; and/or a minimum change in orientation of the smartphone.

Optionally, the message is only communicated if the smartphone determines that the detection event has occurred for a predetermined minimum timespan.

Typically, the message is communicated from the smartphone using, at least in part, a mobile broadband protocol, for example selected from one or more of: W-CDMA, HSPA, CDMA2000, GSM EDGE-Evolution, HSPA+, LTE, and LTE-Advanced.

Preferably, the program is further configured to cause the smartphone to: generate additional information in response to determining the detection event; and include the additional information into the message. The additional information may comprise GPS location information.

Optionally, the smartphone is further configured to: in response to receiving from a receiver device or an other device via a public mobile telecommunications communication network an enquiry message: capture image data from a camera of the smartphone; and communicate, at least in part via a public mobile telecommunications communication network, to the receiver device or other device the captured image data.

The smartphone may be further configured to: receive auxiliary information from at least one local auxiliary device; and communicate the auxiliary information to at least one receiver device and/or at least one other device. Optionally, the smartphone is configured to receive information related to the animal from at least one auxiliary device. Also optionally, the smartphone is configured to receive information related to the environment of the animal from at least one auxiliary device. The smartphone may be further configured to process the auxiliary information. Optionally, at least one auxiliary device is selected from: heart rate monitor; ambient temperature sensor; animal temperature sensor; and auxiliary camera.

Optionally, the smartphone is further configured to: receiving auxiliary information from one or more local auxiliary devices; determining that the received auxiliary information meets predetermined auxiliary criteria associated with the auxiliary information; and in response, communicating, at least in part via a public mobile telecommunications communication network, to one or more receiver devices a message indicative of identification of the predetermined auxiliary criteria being met.

According to another aspect of the present invention, there is provided a smartphone according to the above aspect, and a receiver device for receiving the message communicated by the smartphone. Optionally, the receiver device is configured for displaying an alert to a user of the receiver device in response to receiving the message from the smartphone.

According to another aspect of the present invention, there is provided a case for securing a smartphone to an animal, the case comprising attachment means for attaching the case to a halter of the animal, and a backing layer and a front layer that, when secured to one another, define a pocket for holding the smartphone, wherein the front layer includes one or more open portions and/or one or more transparent windows. The animal may be a mare.

The case may be formed integrally, or may be permanently attached, to the halter. Alternatively, the case may be formed separately to the halter and configured to be removably attached to the halter.

Optionally, the attachment means comprises: a strip extending from a first surface of the backing layer; and a fastener configured for securing a free portion of the strip to the first surface, such that the strip and the backing layer encompass a portion of the halter thereby attaching the case to the halter and wherein the first surface is opposite to the pocket. The fastener may comprise complementary Velcro features on the first surface and the strip.

Optionally, the backing layer and/or front layer are at least partially formed of neoprene. The backing layer may comprise a webbing.

The inventors have discovered that standard features of conventional smartphones such as smartphones can, advantageously, be utilised for monitoring pregnant female horses (mares) in order to determine when the mare is foaling. Such smartphones cannot inherently monitor mares, and require specific configuration to do so. A mare will lay down “flat out” to give birth. Being conventional in nature (i.e. not originally designed for the specific application of the invention), the smartphone is modified with program code in order to identify when the mare has lain down, and to take appropriate action in the form of communicating the message to the receiver.

According to another aspect of the present invention, there is provided a method of operating a monitoring device for monitoring an animal, the method comprising the steps of: monitoring a position and orientation of the monitoring device; determining that a detection event indicative of the animal lying down from a standing position has occurred corresponding to a change in position and/or a change in orientation satisfying a predetermined criteria; and in response, communicating, at least in part via a public mobile telecommunications communication network, to one or more receiver devices a message indicative of identification of a detection event. The animal may be a mare.

According to another aspect of the present invention, there is provided a monitoring device, comprising a processor interfaced with a wireless communication module, a position sensor and/or an orientation sensor, the monitoring device including a program configured to cause the monitoring device to: monitor a position and orientation of the smartphone when attached to an animal; determine that a detection event indicative of the animal lying down from a standing position has occurred corresponding to a change in position and/or a change in orientation satisfying a predetermined criteria; and in response, communicate, at least in part via a public mobile telecommunications communication network, to one or more receiver devices a message indicative of identification of a detection event. The animal may be a mare.

According to another aspect of the invention, there is provided a method of operating a smartphone for monitoring an animal, the method comprising the steps of: receiving from a receiver device or an other device via a public mobile telecommunications communication network an enquiry message; and in response: capturing image data from a camera of the smartphone; and communicating, at least in part via a public mobile telecommunications communication network, to the receiver device or other device the captured image data. The animal may be a mare.

According to another aspect of the present invention, there is provided a smartphone, comprising a processor interfaced with a wireless communication module and a camera, the smartphone including a program configured to cause the smartphone to: in response to receiving from a receiver device or an other device via a public mobile telecommunications communication network an enquiry message: capture image data from a camera of the smartphone; and communicate, at least in part via a public mobile telecommunications communication network, to the receiver device or other device the captured image data.

According to another aspect of the present invention, there is provided a computer program comprising code configured to implement the method of the above aspects when executed by a processor of a smartphone.

As used herein, the word “comprise” or variations such as “comprises” or “comprising” is used in an inclusive sense, i.e. to specify the presence of the stated features but not to preclude the presence or addition of further features in various embodiments of the invention.

### BRIEF DESCRIPTION OF THE DRAWINGS

In order that the invention may be more clearly understood, embodiments will now be described, by way of example, with reference to the accompanying drawing, in which:

FIG. 1 shows a smartphone in communication with one or more receiver devices;

FIG. 2 shows a schematic of a smartphone;

FIG. 3 shows a standing horse with the smartphone attached to its neck;

FIG. 4 shows a method of operating the smartphone;

FIG. 5 shows an auxiliary device in communication with the smartphone;

FIG. 6 shows a case for holding the smartphone; and

FIG. 7 shows a method for operating the smartphone to communicate a message in response to a reading my a local auxiliary device.

### DESCRIPTION OF EMBODIMENTS

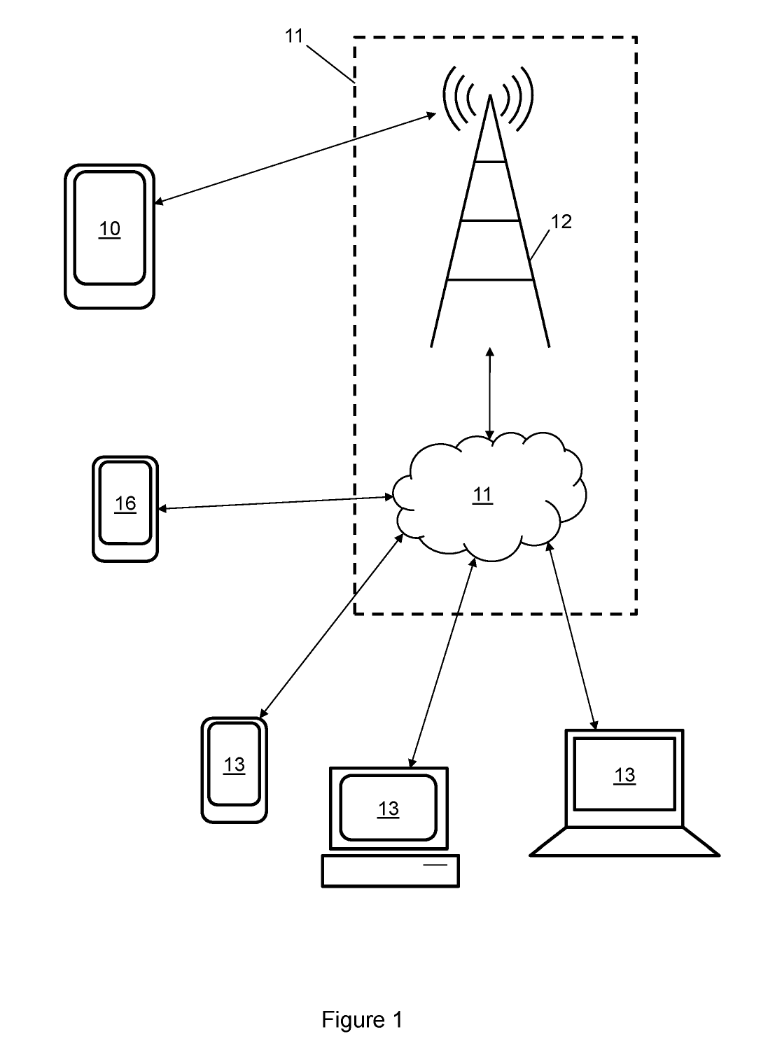

Referring to FIG. 1, in an embodiment, a monitoring device being a smartphone 10 is shown in wireless communication with public mobile telecommunications network (“mobile network”) 11. Through this mobile network 11, the smartphone 10 can communicate with one or more receiver devices 13. The receiver devices 13 can be a mobile phone (e.g. a smartphone), portable computer, tablet, PC, or any other device suitable for receiving communications from the smartphone 10. For convenience, unless stated otherwise, reference is made to a single receiver device 13 (however, this is not intended to be limiting).

The smartphone 10 communicates with the mobile network 11 via a physical infrastructure comprising cellular transponders 12 to which the smartphone 10 communicates wirelessly (i.e. utilising RF communications). The mobile network 11 allows for data communication and typically voice communication (together referred to as “communication”) between the smartphone 10 and the receiver device 13. The communication can occur solely within a mobile network 11 operated by a single telecommunications network provider, between two or more mobile networks 11 each operated by a unique telecommunications network provider, and optionally via a general data network such as the Internet.

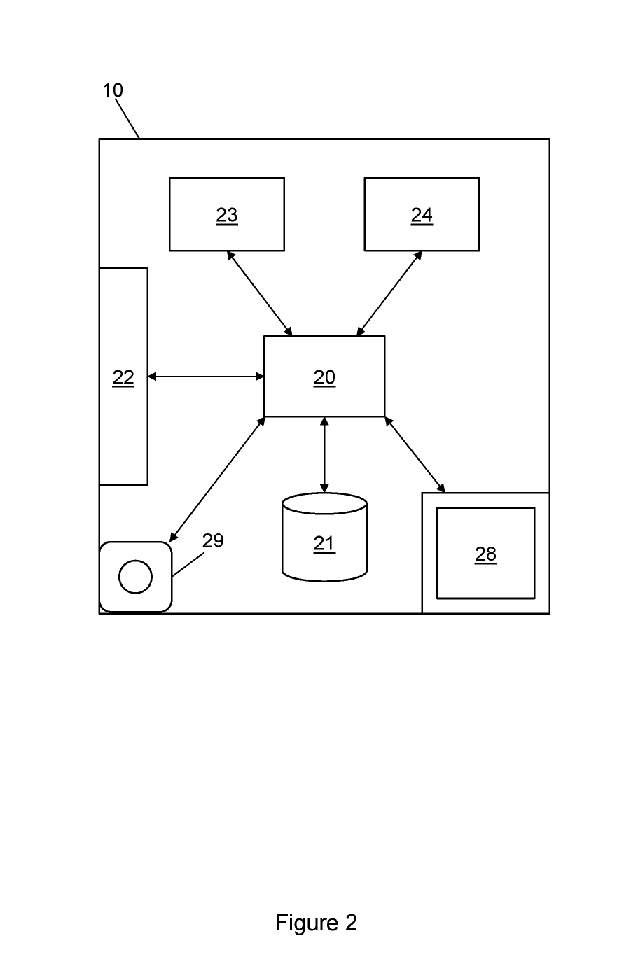

Referring to FIG. 2, the smartphone 10 comprises a processor 20 interfaced with a memory 21, a wireless communication module 22, a position sensor 23, and an orientation sensor 24. For convenience, the positon sensor 23 and orientation sensor 24 are collectively referred to herein as sensors 23, 24, unless a distinction is required. The smartphone 10 is a conventional smartphone, such as a smartphone. The memory 21 is configured for storing program instructions and data, and can be written to and read from as required. The processor 20 is configured for executing program instructions stored within the memory 21, and for generating, storing and reading data from within the memory 21. The smartphone 10 also comprises, generally, a display 28 interfaced with the processor 20, configured to present a graphical user interface (GUI). In many instances, the display 28 is touch sensitive, and can therefore be utilised by the smartphone 10 for receiving inputs form a user of the smartphone 10. The smartphone 10 can include button inputs. The smartphone 10 also comprises a battery and an external power supply port (not shown), both of which are suitable for powering the smartphone 10. Typically, the smartphone 10 comprises a camera 29, which is interfaced with the processor 20.

According to embodiments, the smartphone 10 is provided with a program comprising program instructions configured to cause the smartphone 10 to be used for monitoring an animal.

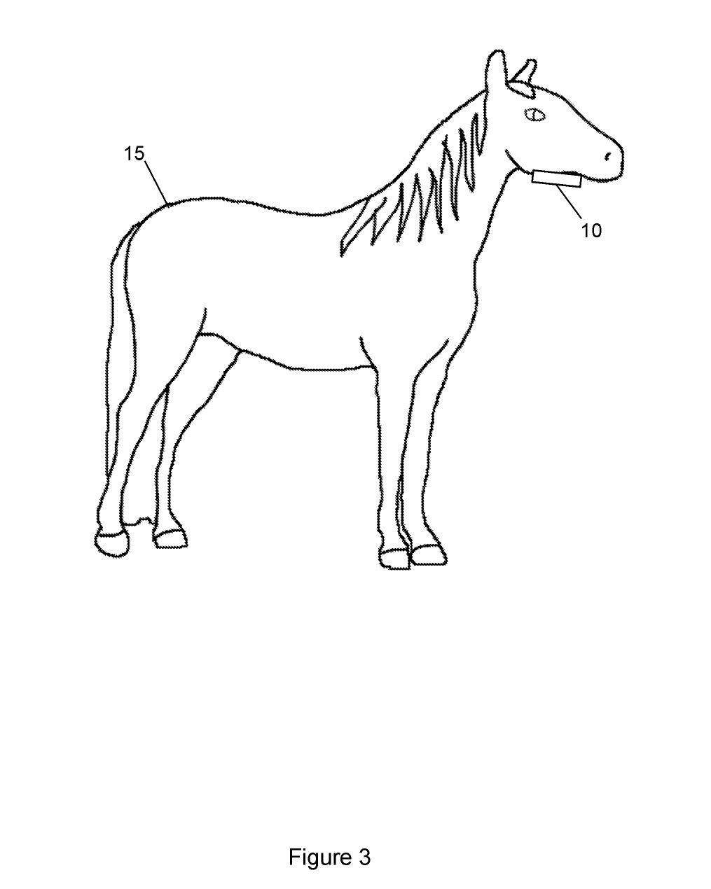

Referring to FIG. 3, an embodiment wherein the smartphone 10 is monitoring a pregnant mare 15 is shown. Specifically, the smartphone 10 is configured, through provision of the program, to identify when the mare 15 is (or at least, is likely to be) lying down, which corresponds to a “detection event”. A mare 15 will usually lie down “flat out” to give birth to a foal. Therefore, in anticipation of birthing, the smartphone 10 is attached to the body of the mare 15. Typically, as shown in FIG. 3, the smartphone 10 will be attached under the chin or jaw of the mare 15.

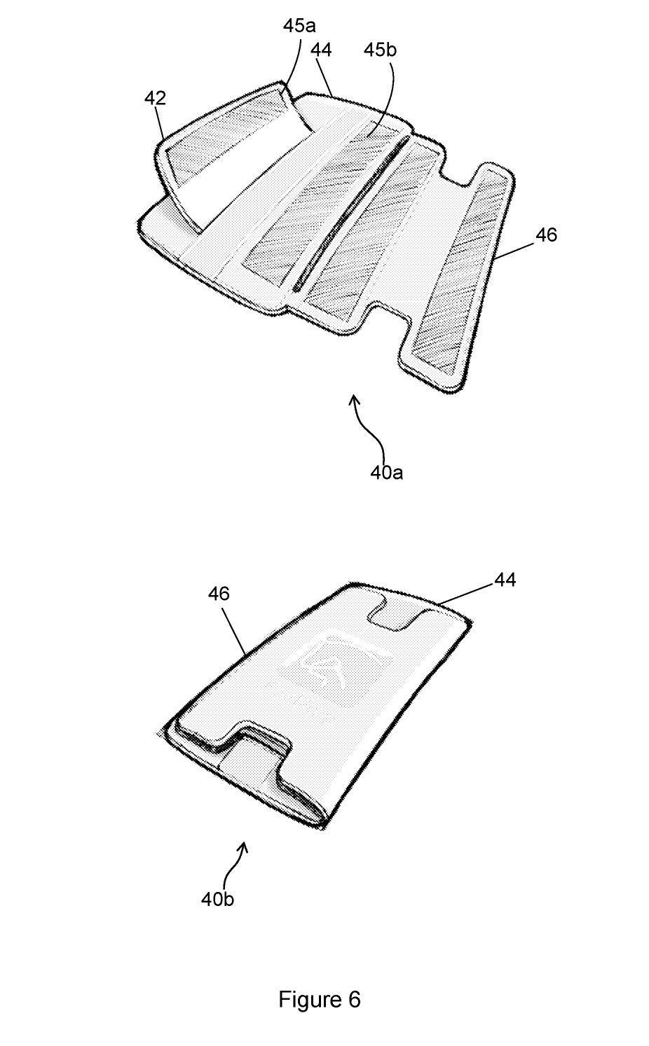

The smartphone 10 may be located within a sleeve of a harness which is itself affixed to the mare 15. In an embodiment, as shown in FIG. 6, a case 40 is provided. Two views of the case 40 are shown (corresponding to an open case view 40a and a closed case view 40b).

The case 40 comprises attachment means for attaching the case to a halter attached to the mare 15. In an embodiment, the attachment means allows for the case 40 to be removably attached to a portion of the halter underneath the head of the mare 15. In the example shown, the attachment means 41 comprises a strip 42 extending from a first surface of a backing layer 44 of the case 40. The strip 42 can run parallel to a length of the case 40 and can be attached to one side of a centre line of the case 40. The case 40 includes a fastener such that the strip 40 can be folded over a portion of the halter of the mare 15 and then secured to the case. In the example shown, the fastener comprises complementary Velcro strips 45a, 45b located on at free portion of the strip 42 and the first surface of the case 40. The fastener thereby allows the case 40 to be secured to a portion of the halter.

In another embodiment, the case 40 is formed integrally with, or is permanently attached, to the halter.

The case 40 is configured to encompass the smartphone 10 sufficiently to securely hold it in place. In an implementation, a pocket is formed between the backing layer 44 and a front layer 46, which is attached to the backing layer 44. The front layer 46 is folded over and secured to the backing layer 45. In the example shown, Velcro strips are used to secure the front layer 46 to the backing layer 45. The smartphone 10 is held within the pocket defined by the backing layer 45 and the front layer 46. As can be seen, the first surface is outward facing with respect to the pocket.

The case 40 is configured such as to not impede the view of a camera 29 of the smartphone 10. In the example, open portions are provided in the front layer 46. Alternatively, a transparent window material can be provided within the front layer 46. The case may include several open portions and/or transparent windows.

The case 40 can comprise a neoprene backing, which can advantageously provide weather protection, and may be relatively comfortable for the mare 15. Multiple layers of neoprene may provide padding against the mare 15. In the example shown, a webbing 48 is provided in the backing layer 44 to provide additional reinforcement.

The program is executed by the processor 20 of the smartphone 10 in order for it to operate its sensors 23, 24 in a manner suitable for identifying the detection event, and to take the appropriate action as a result. Typically, the program will be installed onto the smartphone 10 by a user of the device 10. It is envisaged that the program may be downloaded onto the smartphone 10 from a general repository of programs available via the Internet, for example, from a so-called “app store”. Alternately, other means of loading the program onto the smartphone 10 are predicted, such as “side loading” an application.

The program will typically be initiated by an input (such as pressing an appropriate button (such as an icon) presented on a touch sensitive display 28) by the user of the smartphone 10, for example, immediately before or after attaching the smartphone 10 to the mare 15. The program will continue running until halted by either the user, the occurrence of a predetermined event, or some other event that will halt execution.

The smartphone 10 is now (while executing the program) configured for monitoring the status of the mare 15 in order to identify the detection event (i.e. when the mare 15 is, or is preparing for, foaling).

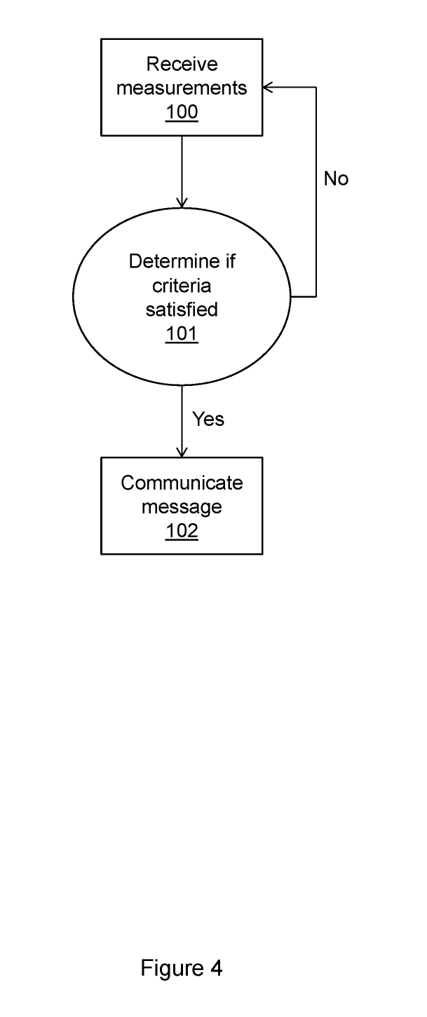

Referring to FIG. 4, the smartphone 10 monitors its own position and/or orientation by receiving outputs from its position sensor 23 (position measurements) and/or its orientation sensor 24 (orientation measurements), at measurement reception step 100. These measurements are typically provided periodically, with the period determined by a required temporal resolution. For example, it may be the case that the smartphone 10 receives measurements on the order of at least every five minutes or so. In another example, measurements are received with a substantially short enough period to be considered continuous.

In an embodiment, the smartphone 10 is configurable such as to only monitor its own position and/or orientation at certain times, for example times during a day, during the night, or any other desired times.

The smartphone 10 compares each received position measurement and/or orientation measurement against predetermined criteria in order to determine, at determination step 101, whether the predetermined criteria is satisfied by the measurement(s). If the predetermined criteria are not satisfied, the method returns to measurement reception step 100. On the other hand, if the predetermined criteria are satisfied, the method proceeds to messaging step 102.

The predetermined criteria are selected such that, in order to be satisfied, it is likely that the mare 15 has changed from a standing state to a lying down state (corresponding to a detection event). The predetermined criteria include a change in orientation being greater than a specified amount and/or a change in position being greater than a predetermined amount. The predetermined criteria can be inputted by the user of the smartphone 10 (e.g. through a touchscreen display 28), or may be set within the program.

The change in position typically corresponds to a change in height roughly equal to the distance between the ground and the smartphone 10 when the smartphone 10 is affixed to the mare 15 and the mare 15 is standing. The predetermined criteria may be different to exactly this height, for example, it may be less than this distance to account for different possible positons of the smartphone 10 relative to the ground depending on how the mare 15 is lying. Typically, the position sensor 23 corresponds to a three-axis accelerometer, from which a change in position is determined based on integrating the output of the accelerometer. The position sensor 23 typically can distinguish between movement corresponding to the mare 15 lying down, and the mare 15 walking along the ground.

The change in orientation typically corresponds to a change in orientation of at least 45 degrees. In practice, the change in orientation should be sufficient to distinguish between the mare 15 in a standing position and in a lying position. The orientation sensor 23 can distinguish between changes in orientation around an axis perpendicular to the ground (i.e. due to the mare 15 turning while remaining standing) and around an axis parallel to the ground (i.e. due to the mare 15 changing from standing to lying down). Typically, the orientation sensor 24 corresponds to a three-axis gyroscope, from which a change in orientation is determined based on integrating the output of the gyroscope.

The position sensor 23 and/or orientation sensor 24 typically is able to identify “down”, or rather, the direction of gravitational pull. It is understood that the position sensor 23 and the orientation sensor 24 may be features of the same component (e.g. silicon chip) of the smartphone 10.

In an embodiment, the smartphone 10 is also configured to ensure that the sensor(s) 23, 24 are producing measurements that satisfy the predetermined criteria for at least a specified time. In this way, the chance of spurious measurements not associated with the mare 15 lying down resulting in the smartphone 10 proceeding to messaging step 102 is reduced. For example, as the smartphone 10 may be affixed under the jaw or chin of the mare 15, movement of the head of the mare 15, such as shaking, may briefly result in measurements satisfying the predetermined criteria.

Referring now to messaging step 102, the smartphone 10 is configured to communicate a message to the receiver device 13. The message comprises information identifying the smartphone 10. The receiver device 13 is typically specified by the user of the smartphone 10 during an initial configuration of the smartphone 10. The receiver device 13 can be specified, for example, by entering a mobile phone number of the receiver device 13, an email address associated with the receiver device 13, or through entering of some other suitable identification information associated with the receiver device 13.

The message is communicated via the public mobile telecommunications network 11. In an embodiment the message is communicated as an SMS (Short Messaging Service) text message. In another embodiment, the message is communicated via a mobile broadband protocol such as W-CDMA, HSPA, CDMA2000, GSM EDGE-Evolution, HSPA+, LTE, LTE-Advanced, etc. It is envisaged that a modification using a voice channel of a smartphone 10 could also be utilised to communicate the message.

The mobile network 11 provides a suitable means for communicating between the smartphone 10 and the receiver device 13 without requiring onsite installation of speciality equipment for communication with the smartphone 10.

The mobile network 11 also provides a wide extent of access; the mare 15 can be monitored, if required, within a wide area without requiring installation of a plurality of speciality equipment to cover the entire area.

The receiver device 13 is configured to generate an alert, for example a visual alert on a screen of the receiver device 13 and/or an audible alert through a speaker of the receiver device 13, in response to receiving the message from the smartphone 10. The alert is configured for informing a user of the receiver device 13 that the detection event has occurred; that is, the mare 15 is now lying down. Receipt of the message is thereby utilised to inform a user of the receiver device 13 that the smartphone 10 has identified the detection event.

In an embodiment, the message comprises additional information that can then be displayed to the user of the receiver device 13. Additional information here means information generated by the smartphone 10. The additional information can include, for example, location information (such as GPS coordinates), time at which the detection event was identified, etc. The additional information is generated by the smartphone 10, where necessary, utilising additional modules of the device 10 (such as a GPS module).

In an embodiment, the smartphone 10 is configured to communicate image data in response to identifying the detection event. The image data is typically communicated to the receiver device 13, however, the smartphone 10 can also, or alternatively, be configured to communicate the image data to one or more other devices 16 (as shown in FIG. 1), being devices that do not receive the message (as with the receiver device 13, for convenience, reference herein is to one other device 16). The image data is generated by a camera 29 of the smartphone 10. In this embodiment, it is typically desirable to position the smartphone 10 on the mare 15 in a manner that will enable useful capture of image data. Optionally, the image data is communicated in response to the programming device 10 receiving an instruction to generate and communicate the image data. For example, such an instruction can be received from the receiver device 13 or the other device 16.

In an implementation, the image data is a single photograph obtained in response to identifying the detection event. The photograph may be obtained after a predetermined interval from identifying the detection event. In another implementation, the image data is a series of photographs captured at predetermined intervals (i.e. periodically). For example, a photograph may be captured every 1, 5, 10, or 30 minutes, or at any other suitable period.

In yet another implementation, the image data corresponds to video data. Video data is distinguished from periodic photographs as it typically has a much higher capture rate (of the order of 1 second or less) and further will typically utilise different functionality of the smartphone 10, for example, a video capture subroutine rather than a still image capture subroutine. The video data can be captured at a fixed resolution. Optionally, a user can cause the receiver device 13 and/or other device 16 to communicate an instruction to the smartphone 10 to select one of an available plurality of resolutions.

In an embodiment, the receiver device 13 and/or other device 16 are configured to receive a user input selecting an image data mode. The image data mode can correspond, for example, to a photograph mode or a video mode. On receiving a selection by the user, the receiver device 13 and/or other device 16 communicates an image data mode message to the smartphone via the mobile network 11. The smartphone 10 is configured to obtain image data corresponding to the image data mode specified in the image data mode message.

A mare 15 may begin foaling at anytime, including when available ambient light is low. In an implementation, the smartphone 10 further comprises a light. The light can be automatically activated when the camera 29 is being utilised. However, it is preferred to allow a user to cause the light to activate and deactivate (for example, the mare 15 may be stressed by the light). In this case, the receiver device 13 and/or other device 16 are configured to allow a user input to select a “light ON” or “light OFF” mode for the smartphone 10. In response to the selection, the receiver device 13 and/or other device 16 communicates via the mobile network 11 a light instruction message to the smartphone 10. The light instruction message comprises an instruction to activate or deactivate the light of the smartphone 10, as required. In response to receiving the light instruction message, the smartphone 10 either activates or deactivates the light as appropriate. Optionally, the smartphone 10 is configured to turn the light of when the camera 29 is no longer being utilised and/or after a predetermined time has elapsed.

The image data is communicated to the receiver device 13 and/or other device 16 via the mobile network 11. The smartphone 10 can be configured to compress the image data before communication in order to reduce the bandwidth required.

Furthermore, in an implementation, the smartphone 10 is configured to obtain audio data (this may be automatic with capturing video data or separate). Audio data can be communicated to the receiver device 13 and/or other device 16.

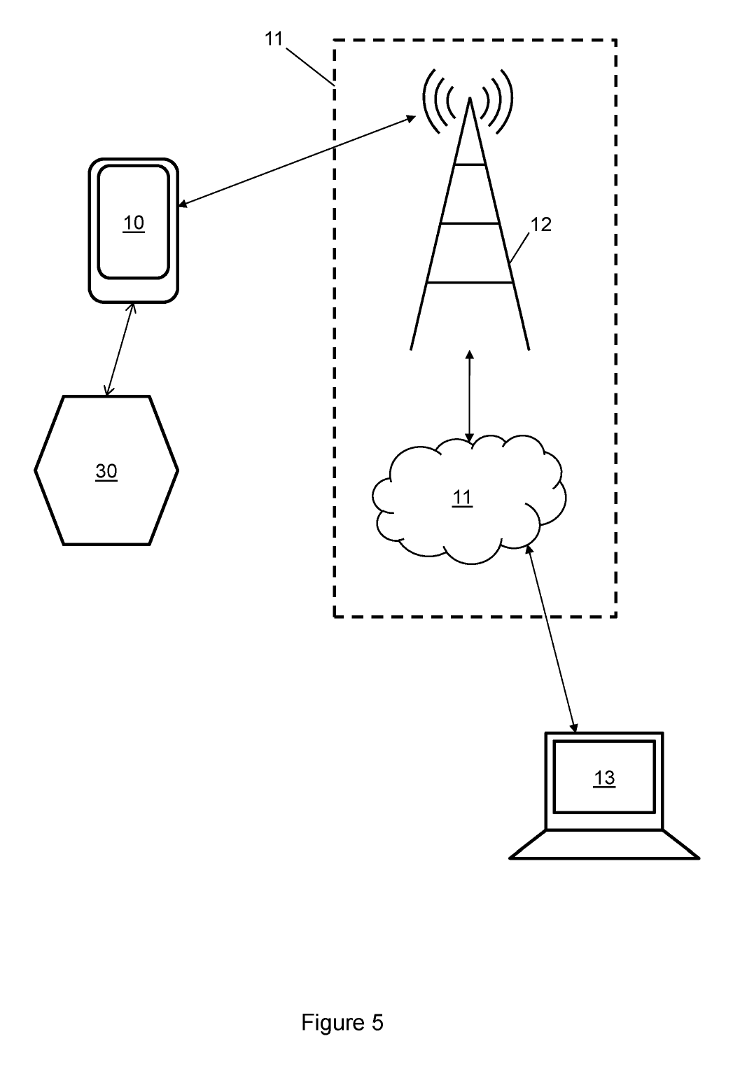

In an embodiment, as shown in FIG. 5, the smartphone 10 is in communication with a local auxiliary device 30. The smartphone 10 can be configured for wireless or wired communication with the local auxiliary device 30, as required. The local auxiliary device 30 provides the smartphone 10 with auxiliary information related to the mare 15 and/or the environment of the mare 15.

In one example, the local auxiliary device 30 is a heart monitor attached to the mare 15. The heart monitor is configured to provide information on the functioning of the mare's heart, such as current heart rate, to the smartphone 10. In another example, the local auxiliary device 30 is a temperature sensor for measuring the ambient temperature in the vicinity of the mare 15, and is not attached to the mare 15. In another example, the local auxiliary device 30 is a mare temperature sensor for measuring the body temperature of the mare 15, and is attached to the mare 15. In yet another example, the auxiliary device 30 is an auxiliary camera located remotely from the mare 15 and configured for capturing images or video of the mare 15. Generally, there may be one or more auxiliary devices 30 in communication with the smartphone 10. There can be auxiliary devices 30 configured for providing different auxiliary information. The auxiliary devices 30 include “off-the-shelf” devices and devices specifically designed for use with monitoring a mare 15 to assist with identifying and monitoring foaling.

The auxiliary information can be communicated to the receiver device 13 and/or other device 16 via the mobile network 11, for example in association with the message. The smartphone 10 can be configured to process the auxiliary information before communication in order to reduce the bandwidth required. Generally, the auxiliary information can be communicated continuously, periodically, or in response to an instruction received at the smartphone 10 from the receiver device 13 and/or other device 16 via the mobile network 11.

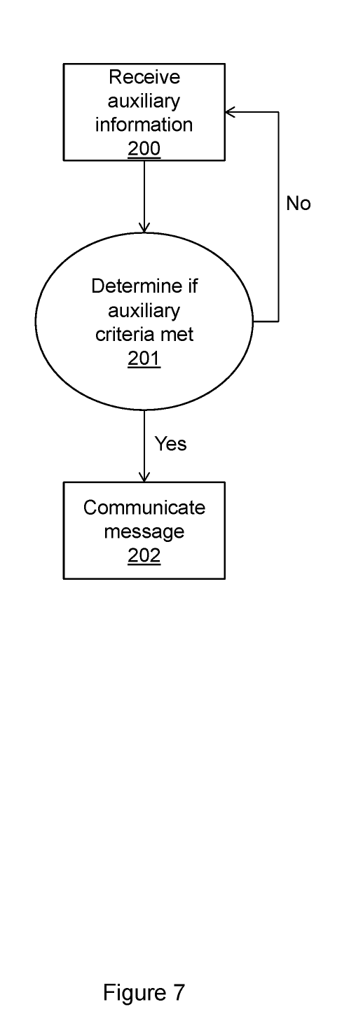

According to an embodiment, with reference to FIG. 7, the smartphone 10 is configured to communicate a message to the receiver device 13 in response to reception of auxiliary information from one or more local auxiliary devices 30 meeting predetermined auxiliary criteria (the criteria may comprises one criterion—the term “criteria” is used to encompass one or more criteria). Typically, the predetermined auxiliary criteria are set by a user of the smartphone 10. The predetermined auxiliary criteria typically specify a status of a local auxiliary device 30 associated with the auxiliary criteria. For example, predetermined auxiliary criteria associated with a heart monitor may correspond to a heart rate reading provided to the smartphone 10 exceeding a predetermined maximum heart rate (or, in another example, a heart rate reading below a predetermined minimum heart rate), optionally for a predetermined period of time (e.g. 10 seconds).

The smartphone 10 therefore receives auxiliary information from the one or more local auxiliary devices 30 at receive auxiliary information step 200. The smartphone 10 compares each received instance of auxiliary information against the predetermined auxiliary criteria associated with the auxiliary information in order to determine, at auxiliary determination step 201, whether the predetermined auxiliary criteria are met by the auxiliary information. If the predetermined auxiliary criteria are not met, the method returns to receive auxiliary information step 200. On the other hand, if the predetermined auxiliary criteria are met, the method proceeds to auxiliary messaging step 202. In an embodiment, two or more predetermined auxiliary criteria, each associated with a different local auxiliary device 30, must be met before proceeding to auxiliary messaging step 202.

Referring now to auxiliary messaging step 202, the smartphone 10 is configured to communicate a message to the receiver device 13 in a similar manner described with respect to FIG. 4. The message can be equivalent to the message described in FIG. 4, or may comprise separate information for example identifying which predetermined auxiliary criteria cause the sending of the message.

In an embodiment, the receiver device 13 and/or other device 16 can communicate, for example in response to a user input, an enquiry message to the smartphone 10, typically via the mobile network 11. The enquiry message comprises an instruction for the smartphone 10 to communicate with the receiver device 13 and/or other device 16. Typically, the enquiry message provides an instruction to the smartphone 10 to communicate image data and/or audio data to the receiver device 13 and/or other device 16. Optionally, the enquiry message can comprise an instruction for the smartphone 10 to communicate auxiliary information to the receiver device 13 and/or other device 16, in applicable embodiments. In this way, a user of the receiver device 13 and/or other device 16 can receive information relating to the mare 15 without a detection event having occurred.

In an embodiment, the smartphone 10 can be instructed to “sleep” for a period of time. The instruction can be provided through an action of a user, for example, interacting with an input of the smartphone 10 or by utilising a receiver device 13 or other device 16 and communicating, via the mobile network 11, the instruction to the smartphone 10.

Although the present disclosure is primarily directed towards the monitoring of a pregnant mare 15, the embodiments described herein may be applicable to monitoring of other animals. For example, other animals may be monitored in which a change in position (such as lying down) corresponds to giving birth. Also, another example is where a change in position and/or orientation is unexpected, and may be a result of an animal being unwell—for example, an unwell animal may change position (lie down) at a time of day when it would normally be expected not to do so. Furthermore, a user can cause an enquiry message to be communicated to the smartphone 10 when attached to an animal generally in order to receive image data and/or audio data at receiver device 13 and/or other device 16.

According to an embodiment, instead of utilising a smartphone 10, a dedicated monitoring device is utilised. The dedicated monitoring device comprises a processor and other features similar to that of a smartphone 10 (although, for example, it may not comprise a display). However, rather than running software installed onto a smartphone 10 to enable the functionality described herein with reference to a smartphone 10, the dedicated monitoring device comprises preconfigured program instructions (such as located in ROM, EEPROM, Flash memory, etc) configured to cause the processor to implement the functionality required of the monitoring device as described with reference to a smartphone 10. Thus, reference herein to “monitoring device” is to be construed as including either or both of a smartphone 10 and a dedicated monitoring device, unless stated otherwise, and the description is relation to use of a smartphone 10 should be understood as applying to a dedicated monitoring device, except where such description relates to features and functionality unique to a smartphone 10.

Further modifications can be made without departing from the spirit and scope of the specification.

1-64. (canceled)

65. A method of operating a smartphone for monitoring an animal, the method comprising the steps of:
monitoring a position and/or orientation of the smartphone;
determining that a detection event indicative of the animal lying down from a standing position has occurred corresponding to a change in position and/or a change in orientation satisfying a predetermined criteria; and
in response, communicating, at least in part via a public mobile telecommunications communication network, to one or more receiver devices a message indicative of identification of a detection event.

66. The method as claimed in claim 65, the method in response to determining that the detection event has occurred further comprising the steps of the smartphone:
obtaining image data; and
communicating said image data to the receiver device and/or one or more other devices.

67. The method as claimed in claim 66, further comprising the step of activating a light of the smartphone.

68. The method as claimed in claim 67, wherein the light is activated such that the image data is captured when the light is activated.

69. The method as claimed in claim 65, the method in response to determining that the detection event has occurred further comprising the steps of the smartphone:
obtaining audio data; and
communicating said audio data to the receiver device and/or one or more other devices.

70. The method as claimed in claim 65, further comprising the steps of:
configuring the smartphone with at least the predetermined criteria; and
initiating the smartphone in response to a user input, and then subsequently monitoring the position and orientation of the smartphone.

71. The method as claimed in claim 65, wherein the predetermined criteria include:
a minimum change in height of the smartphone; and/or
a minimum change in orientation of the smartphone.

72. The method as claimed in claim 65, further comprising the steps of:
receiving, at the smartphone, auxiliary information from at least one local auxiliary device; and
communicating the auxiliary information to at least one receiver device and/or at least one other device.

73. A smartphone, comprising a processor interfaced with a wireless communication module, a position sensor and/or an orientation sensor, the smartphone configured to execute a program configured to cause the smartphone to:
monitor a position and/or orientation of the smartphone when attached to an animal;
determine that a detection event indicative of the animal lying down from a standing position has occurred corresponding to a change in position and/or a change in orientation satisfying a predetermined criteria; and
in response, communicate, at least in part via a public mobile telecommunications communication network, to one or more receiver devices a message indicative of identification of a detection event.

74. The smartphone as claimed in claim 73, wherein the program is further configured to cause the smartphone to:
obtain image data from a camera of the smartphone; and
communicate said image data to the receiver device and/or one or more other devices.

75. The smartphone as claimed in claim 74, further comprising a light, and wherein the program is configured to activate and deactivate the light.

76. The smartphone as claimed in claim 75, wherein the light is activated such that the image data is captured when the light is activated.

77. The smartphone as claimed in claim 73, wherein the program is further configured to cause the smartphone to:
obtain audio data; and
communicate said audio data to the receiver device and/or one or more other devices.

78. The smartphone as claimed in claim 73, wherein the program is further configured to cause the smartphone to:
configure the smartphone with at least the predetermined criteria; and
initiate the smartphone in response to a user input, and then subsequently monitor the position and orientation of the smartphone.

79. The smartphone as claimed in claim 73, wherein the predetermined criteria include:
a minimum change in height of the smartphone; and/or
a minimum change in orientation of the smartphone.

80. The smartphone as claimed in claim 73, further configured to:
receive auxiliary information from at least one local auxiliary device; and communicate the auxiliary information to at least one receiver device and/or at least one other device.

81. The system for monitoring a mare, comprising a smartphone according to claim 9, and a receiver device for receiving the message communicated by the smartphone.

82. A case for securing a smartphone to an animal, the case comprising attachment means for attaching the case to a halter of the animal, and a backing layer and a front layer that, when secured to one another, define a pocket for holding the smartphone, wherein the front layer includes one or more open portions and/or one or more transparent windows.

83. The case as claimed in claim 82, wherein the attachment means comprises:
a strip extending from a first surface of the backing layer; and
a fastener configured for securing a free portion of the strip to the first surface,
such that the strip and the backing layer encompass a portion of the halter thereby attaching the case to the halter and wherein the first surface is opposite to the pocket.

84. The case as claimed in claim 82, wherein the backing layer and/or front layer are at least partially for lied of neoprene.
